(19) United States
(12) Patent Application Publication
(10) Pub. No.: US 2016/0287784 A1
(43) Pub. Date: Oct. 6, 2016

(54) PUMP DELIVERY CALCULATION SYSTEMS AND METHODS

(71) Applicant: Smiths Medical ASD, Inc., Plymouth, MN (US)

(72) Inventors: Scott Straw, Blaine, MN (US); William Van Dyke, Inver Grove Heights, MN (US)

(21) Appl. No.: 15/035,877

(22) PCT Filed: Nov. 3, 2014
(86) PCT No.: PCT/US2014/063683

§ 371 Date: May 11, 2016

Related U.S. Application Data
(60) Provisional application No. 61/903,161, filed on Nov. 12, 2013.

(51) Int. Cl. A61M 5/172; G06F 19/00; A61M 5/142

(52) U.S. Cl. CPC A61M 5/172; A61M 5/14236; A61M 5/14228; G06F 19/3406; A61M 2005/14208; A61M 2205/70; A61M 2205/52; A61M 2205/8206

(57) ABSTRACT

An infusion pump system includes at least one reservoir having a reservoir volume, and an infusion pump including a pumping mechanism operably coupled to the at least one reservoir, a processor including a memory and configured to control operation of the pumping mechanism, and a calculation module configured to determine at least one characteristic about the at least one reservoir.

### TECHNICAL FIELD

Subject matter hereof relates generally to infusion pumps, and more particularly, to systems and methods for the calculation of delivery metrics for infusion pumps.

### BACKGROUND

Infusion pumps are extremely useful medical devices for providing prescribed fluids and drug and other therapies to patients. For example, medications such as antibiotics, chemotherapy drugs, and pain relievers are commonly delivered to patients via an infusion pump, as are nutrients and other supplements. Infusion pumps have been used in hospitals, nursing homes, and in other short-term and long-term medical facilities, as well as for in-home care. Infusion pumps can be particularly useful for the delivery of medical therapies requiring an extended period of time for their administration. There are many types of infusion pumps, including large volume, patient-controlled analgesia (PCA), elastomeric, syringe, enteral, and insulin pumps. Infusion pumps are typically useful in various routes of medication delivery, including intravenously, intra-arterially, subcutaneously, intraperitoneally, in close proximity to nerves, and into an intraoperative site, epidural space or subarachnoid space. Infusion pumps are generally coupled to one or more reservoirs configured to hold or store the medication to be infused, such as a syringe, cassette, IV bag, or other self-contained fluid source.

Some traditional infusion pump systems can display (and sometimes compute) a value representing the time remaining until the current reservoir is expended. This value (reservoir volume time remaining) is often calculated based on a simplistic linear approximation. In this regard, infusion pumps typically consider only the current reservoir volume remaining and the current delivery rate in calculating a time remaining. An approximation of the time remaining for the reservoir is made by dividing the delivery rate into the reservoir volume. This approximation, however, does not account for the various phases of infusate delivery that are possible on an infusion pump, nor other events that can occur during delivery.

For example, the various phases of delivery on an infusion pump can include: a loading dose, which can last for a given duration (and volume) to complete; a main dose, which can last for a different duration (and volume) to complete and which might reach a volume limit in a given duration; and a Keep Vein Open (KVO) rate, which often is a different rate than the loading or main doses. Depending on the reservoir volume remaining at any given time of operation, the pump could reach empty at any time during one of these phases (or other potential phases). A simple linear approximation thus would not satisfactorily account for such variations in infusion pump phase delivery.

Therefore, there is a need for an infusion pump that is readily able to account for the various steps or phases of an infusion or planned infusion(s) (regardless of infusion type or method, such as a “tapered infusion”) and precisely determine the reservoir volume time remaining, among other metrics.

### SUMMARY

Embodiments described or otherwise contemplated herein substantially meet the aforementioned needs; for example, accounting for various infusion steps or phases in determining the reservoir volume time remaining, among other metrics. In an embodiment, an infusion pump is configured to take into account the various steps or stages of the infusion or planned infusion(s), how long each step or stage will take, and combine the result in order to calculate a reservoir volume time remaining. For example, using the programming parameters and variables of the current progress through the infusion(s), an embodiment of the method creates an ordered collection of the current and future phases of an infusion or infusions. The method can then (iteratively, in embodiments) traverse the ordered collection of phases and accumulate the time necessary to execute each phase, stopping at the juncture where the reservoir volume would be expended. In embodiments, other delivery metrics are also calculable, including infusion time remaining and number of reservoirs remaining

In an embodiment, a method of determining a delivery metric for an infusion pump comprises determining at least a plurality of delivery phases of the infusion pump, ordering the determined delivery phases, (iteratively, in embodiments) traversing the ordered delivery phases by evaluating a phase characteristic for each delivery phase against a pump characteristic, and determining the delivery metric based on the traversal of the ordered delivery phases.

In an embodiment, an infusion pump system comprises at least one reservoir having a reservoir volume; and an infusion pump including a pumping mechanism operably coupled to the at least one reservoir, a processor including a memory and configured to control operation of the pumping mechanism, and a calculation module configured to determine at least one characteristic about the at least one reservoir.

In an embodiment, an infusion pump comprises a pumping mechanism operably coupled to at least one reservoir, a pump control subsystem including a processor and a memory and configured to control operation of the pumping mechanism, the operation including a plurality of delivery phases, and a calculation module configured to determine the plurality of delivery phases, order the determined delivery phases, (iteratively, in embodiments) traverse the ordered delivery phases by evaluating a phase characteristic for each delivery phase against a pump characteristic and determining a delivery metric based on the traversal of the ordered delivery phases.

In a feature and advantage of embodiments, a precise determination of an infusion pump reservoir volume time remaining can be provided. By having a precise determination of the reservoir volume time remaining, medical professionals or users attending to or operating the infusion pump can rely on this determination to make medical treatment decisions, such as when to replace a reservoir or when to plan another infusion or other medical treatment. The medical professionals or other user would thereby also have access to other metrics such as infusion time remaining or number of reservoirs remaining. Medical professionals or other users are thereby able to primarily focus on overall patient care rather than pump operation and control.

The above summary is not intended to describe each illustrated embodiment or every implementation of the subject matter hereof. The figures and the detailed description that follow more particularly exemplify these embodiments.

### BRIEF DESCRIPTION OF THE DRAWINGS

Subject matter hereof may be more completely understood in consideration of the following detailed description of various embodiments in connection with the accompanying figures, in which:

FIG. 1A is an example of a perspective view of a syringe type infusion pump, according to an embodiment.

FIG. 1B is an example of a front view of an ambulatory type infusion pump, according to an embodiment.

FIG. 2A is a block diagram of an infusion pump including a reservoir, according to an embodiment.

FIG. 2B is a block diagram of an infusion pump operably coupled to multiple reservoirs, according to an embodiment.

FIG. 3 is a block diagram of an infusion pump system, according to an embodiment.

FIG. 4 is a block diagram of a method of calculating reservoir volume time remaining, according to an embodiment.

FIG. 5 is a class diagram of functions implementing a calculation module, according to an embodiment.

While embodiments are amenable to various modifications and alternative forms, specifics thereof have been shown by way of example in the drawings and will be described in detail. It should be understood, however, that the intention is not to limit subject matter hereof to the particular embodiments described. On the contrary, the intention is to cover all modifications, equivalents, and alternatives falling within the spirit and scope of subject matter hereof in accordance with the appended claims.

### DETAILED DESCRIPTION OF THE DRAWINGS

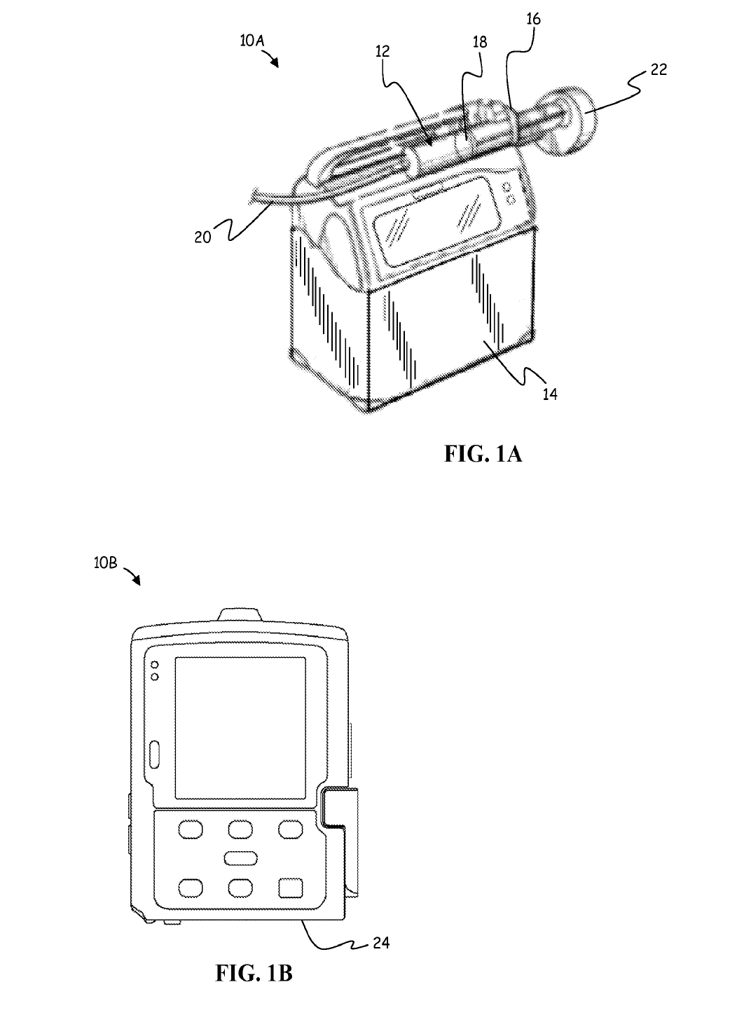

FIGS. 1A and 1B show examples of infusion pumps 10A and 10B, respectively, (also referred to more generally in this disclosure by numeral 10), which can be used to implement embodiments of the systems and methods discussed herein. In general, infusion pump 10A is a syringe-type pump that can be used to deliver a wide range of drug therapies and treatments. Infusion pump 10A includes a pharmaceutical container or syringe 12, which is supported on and secured to housing 14 by clamp 16, respectively. In embodiments, syringe 12 can be separately supplied from pump 10A. In other embodiments, syringe 12 is an integrated component of pump 10A. Syringe 12 includes a plunger 18 that forces fluid outwardly from syringe 14 via infusion line 20 that is connected to a patient. A motor and lead screw arrangement internal to housing 14 of pump 10A cooperatively actuates a pusher or plunger driver mechanism 22, to move plunger 18. In embodiments, a sensor (not shown; which is typically internal to plunger driver mechanism 22), monitors force and/or plunger position in the syringe according to system specifications.

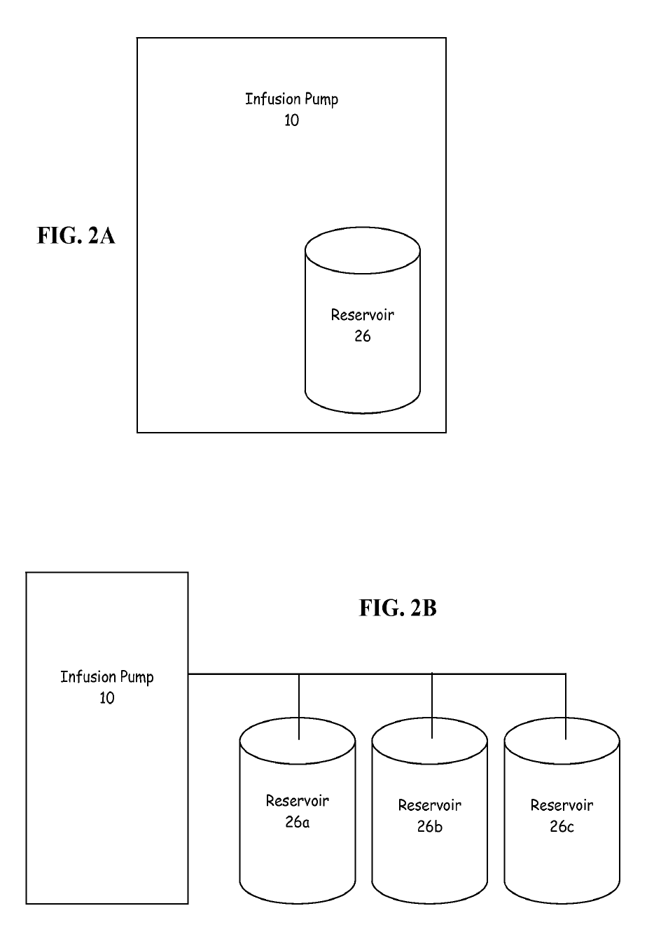

Infusion pump 10B shown in FIG. 1B is an example of an ambulatory pump that can be used to deliver a wide range of drug therapies and treatments. Such ambulatory pumps can be comfortably worn by or otherwise removably coupled to a user for in-home ambulatory care by way of belts, straps, clips or other simple fastening means; and can also be alternatively provided in ambulatory pole-mounted arrangements within hospitals and other medical care facilities. Infusion pump 10B generally includes a peristaltic type infusion pump mechanism that controls the flow of medication from a reservoir (not shown in FIG. 1B) of fluid through a conduit passing along bottom surface 24 of pump 10B. This fluid can be from a cassette reservoir (schematically depicted in FIGS. 2A-2B) that is attached to the bottom of pump 10B at surface 24, or from an IV bag or other fluid source that is similarly connected to pump 10B via an adapter plate (not shown) at surface 24. Specifically, pump 10B uses valves and an expulsor located on bottom surface 24 to selectively squeeze a tube of fluid (not shown) connected to the reservoir to effect the movement of the fluid supplied by the reservoir through the tube and to a patient in peristaltic pumping fashion.

As described above, infusion pump 10 can be operably coupled to one or more reservoirs, syringes, cassettes, IV bags, or other self-contained fluid sources. For example, referring to FIG. 2A, infusion pump 10 can comprise a reservoir 26. Reservoir 26 can be, for example, syringe 12 of infusion pump 10A, or the cassette reservoir attached to the bottom of the pump 10B at surface 24, and can be internal or directly coupled to infusion pump 10. In another embodiment, referring to FIG. 2B, infusion pump 10 can be operably coupled to reservoir 26a, reservoir 26b, and/or reservoir 26c such that reservoirs 26a-26c are external to infusion pump 10. In other embodiments, additional or fewer reservoirs can be operably coupled to infusion pump 10. In other embodiments, infusion pump 10 can include an internal reservoir and additionally be operably coupled to one or more external reservoirs.

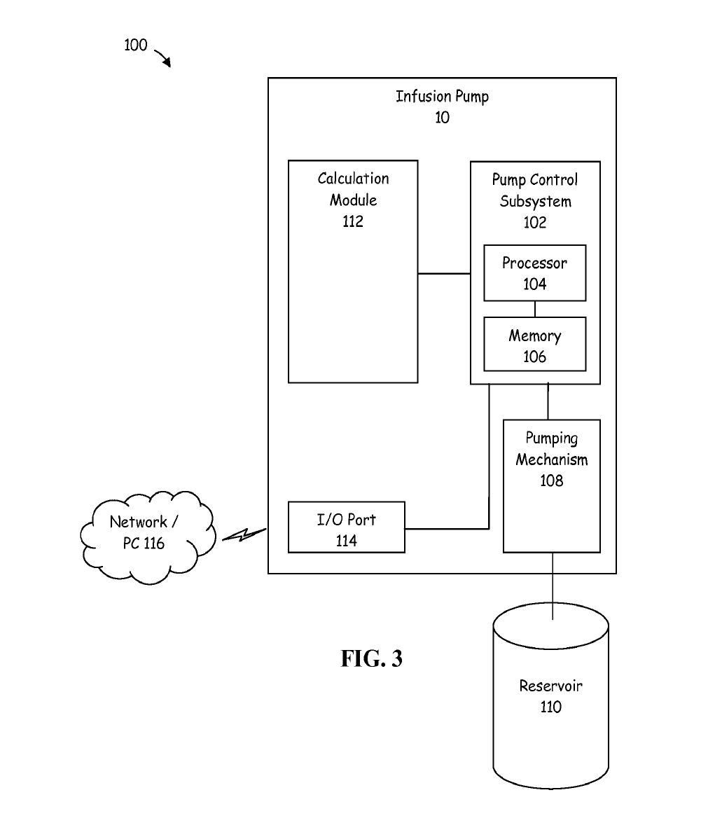

FIG. 3 is a block diagram of various elements of an infusion pump system 100. The system 100 includes an infusion pump 10 having a pump control subsystem 102 including a processor 104 and memory 106 programmable with selected protocols, profiles and other settings for controlling operation of a pumping mechanism 108 such as, e.g., the aforementioned syringe and peristaltic type mechanisms.

Processor 104 can be any suitable programmable device that accepts digital data as input, is configured to process the input according to instructions or algorithms, and provides results as outputs. In an embodiment, processor 104 can be a central processing unit (CPU) configured to carry out the instructions of a computer program. In other embodiments, processor 104 can be an Advanced RISC (Reduced Instruction Set Computing) Machine (ARM) processor or other embedded microprocessor. In other embodiments, processor 104 actually comprises a multi-processor cluster. Processor 104 is therefore configured to perform at least basic selected arithmetical, logical, and input/output operations.

Memory 106 can comprise volatile or non-volatile memory as required by the coupled processor 104 to not only provide space to execute the instructions or algorithms, but to provide the space to store the instructions themselves. In embodiments, volatile memory can include random access memory (RAM), dynamic random access memory (DRAM), or static random access memory (SRAM), for example. In embodiments, non-volatile memory can include read-only memory, flash memory, ferroelectric RAM, hard disk, floppy disk, magnetic tape, or optical disc storage, for example. The foregoing examples in no way limit the type of memory that can be used, as these embodiments are given only by way of example and are not intended to limit subject matter hereof.

It is also to be appreciated and understood that aforementioned calculations could be performed on and/or off the pump by any computational device with access to the delivery parameters and current progress data. As such, for example, a separate data server could be adapted to perform the calculations in two-way communication with, or by receiving one-way data from, the pump.

In embodiments, pumping mechanism 108 is operably coupled to one or more internal or external reservoirs, such as reservoir 110 and as described above with respect to FIGS. 2A and 2B. Infusion pump 10 also includes a calculation module 112 configured to calculate metrics with respect to pump control subsystem 102, and as will be described further below. The infusion pump 10 can further include a USB port, wireless interface, or other appropriate input/output (I/O) interface port 114 for connecting the infusion pump 10 to a network or computer 116 having software configured to interface with the infusion pump 10. Power to the infusion pump 10 is accomplished via an AC power cord and/or internally provided battery.

Referring again to FIG. 3 and calculation module 112, in which calculation module 112 is depicted as discrete from pump control subsystem 102 for ease of explanation, one skilled in the art will readily understand that calculation module 112 can be configured to be implemented, in embodiments, as a discrete subsystem within infusion pump 10 as depicted in FIG. 3. In embodiments, calculation module 112 can be implemented by a processor and memory separate from pump control subsystem 102, processor 104 and memory 106. In other embodiments, calculation module 112 can be implemented as part of pump control subsystem 102 by utilizing processor 104 and memory 106. In still other embodiments, calculation module 112 can be implemented by network/PC 116 such that data relevant to calculation module 112 and reporting tasks can be communicated between network/PC 116 and infusion pump 10 via input/output (I/O) interface port 114.

In operation, a medical professional or user attending to or operating an infusion pump can utilize calculation module 112 in order to make medical treatment decisions. This is particularly useful in a hospital setting, where the medical professional may be monitoring or overseeing dozens of active infusion pumps. For example, the medical professional may be monitoring an infusion for a patient that requires multiple infusion reservoirs such that additional reservoir(s) need to be added during the infusion. In embodiments, the medical professional can rely on outputs of calculation module 112 in order to know when, precisely, the reservoir(s) will need to be changed for that patient. In another example, while that first patient is receiving an infusion, a second patient may be scheduled for a CT scan that requires the second patient to be removed from any infusion devices. In embodiments, the medical professional can rely on outputs of calculation module 112 in order to know when the second patient's infusion is complete (e.g. reservoir empty) and the second patient is ready to proceed to the CT scan. The medical practitioner can therefore precisely plan when to replace the first patient's reservoir in light of the time expected to be required in diverting attention from the first patient and instead attending to the second patient's CT scan.

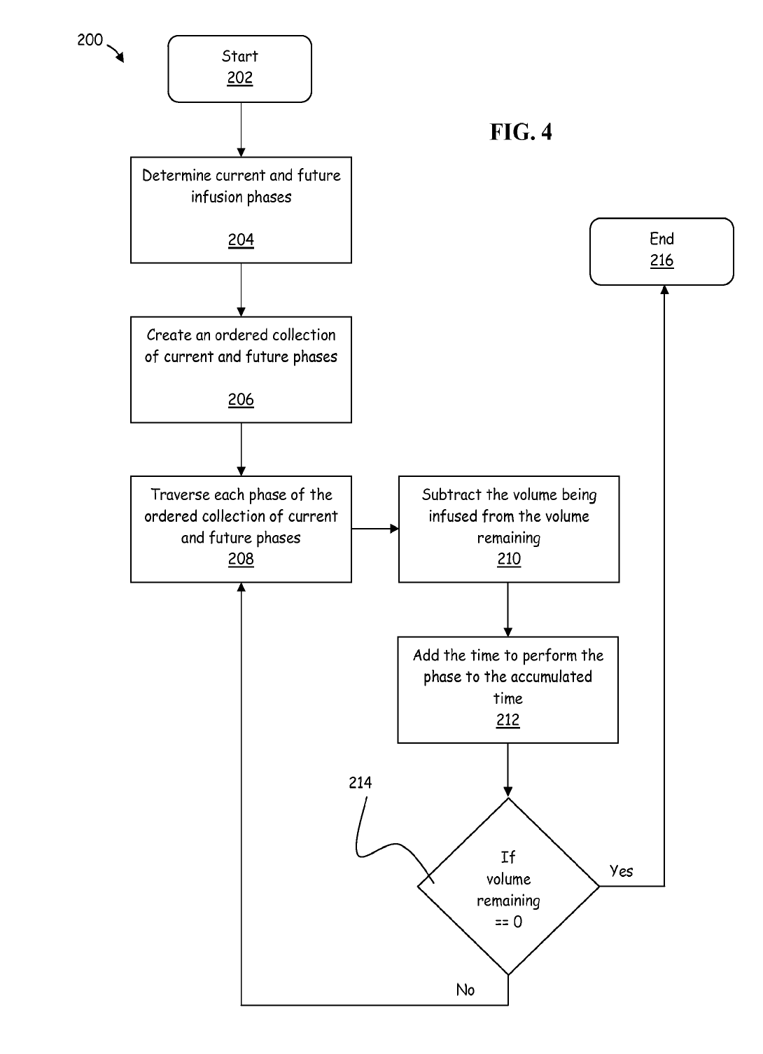

Referring to FIG. 4, a block diagram of an example of a method of calculating reservoir volume time remaining 200 is depicted. Method 200 begins at start 202. In practice, method 200 can begin with an infusion pump being deployed or otherwise programmed for an infusion or series of infusions, and any number of tasks or routines can precede or follow that which is depicted in FIG. 4.

At 204, the delivery phases of the infusion pump are determined In embodiments, the delivery phases can be the current and future delivery phases. In an embodiment, this determination can be made by an evaluation of the infusion pump programming parameters and variables defining the state of progress through the infusion; for example, those stored in memory 106 and implemented by pump control subsystem 102. In other embodiments, the determination of the delivery phases can be made via analysis of devices or systems external to the infusion pump that track the infusion delivery phases for the pump. In embodiments, the determined phases can be stored in an appropriate data structure, such as a table. In other embodiments, an array, hash table, set, tree, record, stack, list, graph, primitive data type, or any other appropriate data structure object can be utilized to store the collection.

At 206, an ordered collection of the delivery phases of the infusion pump that were determined at 204 is created. This ordering can be created by, for example, any appropriate sorting algorithm. In other embodiments, if a phase identification (ID) for the pump programming parameters for the given phases is utilized (assuming the phases comprise phase IDs defined in sequential order), a simple ordering of the phase IDs can be conducted. In embodiments, an ordered table or database can be created at 206, as will be described. In other embodiments, the table created at 204 can be sorted and used as the table for the ordered collection. In other embodiments, an array, hash table, set, tree, record, stack, list, graph, primitive data type, or any other appropriate data structure object can be utilized to store the ordered collection.

In embodiments, 204 and 206 can be combined into a single instance, whereby the ordered collection of delivery phases is created automatically upon determination of all of the delivery phases. For example, if the programming parameters and variables representative of the delivery phases are stored in order such that the determination of 204 creates a sorted list for 206, 204 and 206 are thereby combined into a single task.

At 208 (and more particularly, by the operations of 210-214), each of the phases of the ordered collection of delivery phases is traversed in order, according to an embodiment of an algorithm. In general, method 200 examines a First delivery phase for the pump (as ordered by 206), incorporates one or more items of data for that delivery phase (as defined by the pump operation for that phase), then, identifies a second delivery phase and incorporates one or more items of data for that second delivery phase, and so on.

In embodiments, this traversal can be by iterative, loop-based repetitions of a process or set of processes. In other embodiments, the traversal of 208 of the delivery phases can be implemented by any other appropriate declaration, such as a case statement or series of “if-then-else” statements. Other suitable implementations are, of course, possible and included according embodiments of the invention, depending on the bounds and capabilities of the implementing programming language and underlying hardware.

According to the algorithm of the traversal of 208, for each of the ordered collection of delivery phases, the volume being infused at the instant phase is subtracted from the volume remaining at 210. The volume remaining variable or data structure can be any number of appropriate data structures, as described above with respect to 204 and 206.

At 212, the time duration required to perform the instant volume infusion is added or summed to an aggregated accumulated time. The accumulated time can be an appropriate variable or data structure and can be any number of appropriate data structures.

For example, in embodiments having a delayed start, the duration of time required to complete the remaining portion of the Delayed Start is equal to the Delay Start Time Remaining.

In embodiments having a Loading Dose, the duration of time required to complete the remaining portion of the Loading Dose is equal to:

(Loading Dose Time)*(Loading Dose Volume Remaining)/(Loading Dose Volume).

In embodiments having a Bolus Dose, the duration of time required to complete the remaining portion of the Bolus Dose is equal to:

(Bolus Dose Time)*(Bolus Dose Volume Remaining)/(Bolus Dose Volume).

In embodiments having “per-time” infusions, such as ML/HR, DOSE/DAY, DOSE/HR, DOSE/MIN, DOSE/KG/DAY, DOSE/KG/HR, DOSE/KG/MIN, DOSE/M2/DAY, DOSE/M2/HR, DOSE/M2/MIN, ML/KG/HR, or ML/KG/MIN, the duration of time required to complete the remaining portion of the Main delivery is infinite. This implies that the remaining portion of the current Reservoir Volume Remaining will be delivered during the Main infusion. As a result, the duration of time required to deliver the remaining portion of the current Reservoir Volume Remaining during the Main delivery is:

(Remaining Portion of Reservoir Volume Remaining)/(Main Rate).

In embodiments, some unit conversions are necessary.

In embodiments having VOLUME/TIME, DOSE/TIME, DOSE/KG/TIME, DOSE/M2/TIME infusions, the duration of time required to complete the remaining portion of the Main delivery is equal to:

(Main Dose Volume Remaining)/(Main Rate).

In embodiments, some unit conversions are necessary.

In embodiments having Intermittent Volume Over Time (IVOT) infusions, the duration of time required to complete the remaining portion of the Main delivery is infinite This implies that the remaining portion of the current Reservoir Volume Remaining will be delivered during the Main infusion.

In embodiments having IVOT infusions, the number of complete IVOT cycles it will take (not counting any IVOT cycle currently in progress or any partial cycle at the end) to deliver the remaining portion of the current Reservoir Volume Remaining during the Main delivery is the integer result of the following calculation, rounded down:

((Remaining Portion of Reservoir Volume Remaining)−(Current IVOT Cycle Volume Remaining))/(IVOT Volume per Cycle).

In embodiments, some unit conversions are necessary.

In embodiments having IVOT infusions, when in the delivering portion of the IVOT cycle, and (Remaining Portion of Reservoir Volume Remaining)<=(Current IVOT Cycle Volume Remaining), the duration of time required to deliver the remaining portion of the current Reservoir Volume Remaining during the Main delivery is:

(Remaining Portion of Reservoir Volume Remaining)*(IVOT Cycle Delivery Time)/(IVOT Volume per Cycle)

In embodiments, some unit conversions are necessary.

In embodiments having IVOT infusions, when in the delivering portion of the IVOT cycle, and (Remaining Portion of Reservoir Volume Remaining)>(Current IVOT Cycle Volume Remaining), the duration of time required to deliver the remaining portion of the current Reservoir Volume Remaining during the Main delivery is:

(Current IVOT Cycle Volume Remaining)*(IVOT Time)/(IVOT Volume per Cycle)+

((IVOT Time Between Starts)−(IVOT Time))+(Number of complete IVOT cycles)*(IVOT Time Between Starts)+((Remaining Portion of Reservoir Volume Remaining)−(Current IVOT Cycle Volume Remaining)−(Number of complete IVOT cycles)*(IVOT Volume))*(IVOT Volume)/(IVOT Time)

In embodiments, some unit conversions are necessary.

In embodiments having IVOT infusions, when in the delay portion of the IVOT cycle, the duration of time required to deliver the remaining portion of the current Reservoir Volume Remaining during the Main delivery is:

(Time Between Starts Remaining)+(Number of complete NOT cycles)*(IVOT Time Between Starts)+((Remaining Portion of Reservoir Volume Remaining)−(Number of complete NOT cycles)*(IVOT Volume))*(IVOT Volume)/(IVOT Time)

Note: Some unit conversions are necessary.

In embodiments having KVO infusions, the duration of time required to complete the remaining portion of the KVO delivery is infinite. This implies that the remaining portion of the current Reservoir Volume Remaining will be delivered during the KVO infusion. The duration of time required to deliver the remaining portion of the current Reservoir Volume Remaining during KVO delivery is:

(Remaining Portion of Reservoir Volume Remaining)/(KVO Rate)

In embodiments, if (Remaining Portion of Reservoir Volume Remaining)<(Flush Volume Remaining), then the duration of time required to deliver the remaining portion of the current Reservoir Volume Remaining during Flush delivery is:

(Remaining Portion of Reservoir Volume Remaining)*(Flush Time)/(Flush Volume)

Referring again to FIG. 4, a check of whether the volume remaining equals zero is then conducted, referring to the volume remaining decision point at check 214. If the volume remaining equals zero, the method proceeds to end 216. If however, the volume remaining does not equal zero, method 200 proceeds back to traverse the next ordered current or future phase at 208.

In other embodiments, a volume remaining decision point, similar to check 214, can be implemented in real time within, for example 210 and 212. For example, at 210, if the volume remaining reaches zero upon part of the instant phase volume to be infused being subtracted from the volume remaining, the instant phase remainder volume is not subtracted from the volume remaining (as the volume remaining cannot be a negative value). Similarly, at 212, if the volume remaining reaches zero upon part of the instant phase volume to be infused being subtracted from the volume remaining, only the time representative of the time to perform that part of the phase is added to the accumulated time. In other embodiments, a volume remaining decision point similar to check 214 can be implemented prior to the iterative traversal 210 and 212 (for example, after 214), where appropriate within the algorithm. Of course, one of ordinary skill in the art will readily recognize that the fundamentals of method 200 can be implemented in any number of ways, only limited by the programming language, the underlying hardware, and the imagination of the programmer. FIG. 4 is thereby provided as merely one example of one embodiment of the algorithm.

Method 200 is scalable to multiple pumps and multiple reservoirs. For example, the delivery phases determined by 204 can be of multiple pumps coupled to a patient, each of which can comprise multiple reservoirs. In an embodiment, a single instance of method 200 ran be instantiated for each infusion pump 10 that is coupled to the patient. In other embodiments, if the patient is coupled to multiple infusion pumps 10 that are configured to serially administer infusions one after another, a single instance of method 200 can be utilized, with all of the infusion pump data aggregated using method 200.

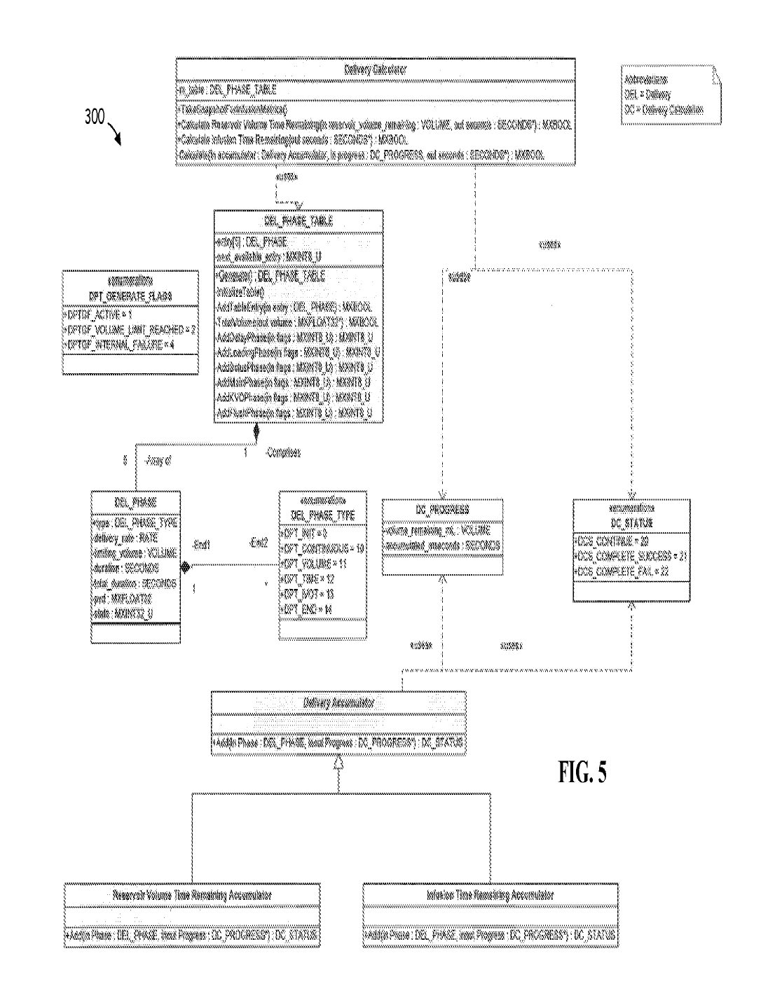

Referring to FIG. 5, the class diagram 300 depicts the functions of, for example, calculation module 112 that implement the method 200 of calculating reservoir volume time remaining, according to an embodiment. For ease of explanation, the class diagram is implemented by object-oriented concepts. However, the implementation is not limited to object-oriented languages or code, and can be implemented or programmed by any suitable language, compiler, or structure.

According to an embodiment, and referring to FIGS. 4-5 and the “Delivery Phase Types” Table 1 below, and for example, 204 and 206 of method 200, an example of operation of the calculation (creation of the Delivery Phase Types Table) is represented in FIG. 5 and by the class, DEL_PHASE_TABLE. A set of functions are called in succession to add phases to the table until all pertinent phases have been added. Table 1 includes DEL PHASE entries. Each DEL_PHASE entry has a type, and depending upon the type, contains information about the delivery rate, volume to be infused, and/or the time duration of the particular phase of delivery. In embodiments, pausing during an infusion or between infusions can also be recorded. The phase entry that is added to the table for the current in-progress phase contains information describing the rest of the phase.

TABLE 1
Delivery Phase Types

| Phase Type | Description | Pertinent Attributes |
|---|---|---|
| DPT_INIT | This type of phase is an “empty | None |
| | entry” in the table. | |
| DPT_CONTINUOUS | Describes a phase of delivery | Delivery Rate |
| | which, if not limited by the | |
| | reservoir volume, would | |
| | continue indefinitely. | |
| DPT_VOLUME | Describes a phase of delivery | Delivery Rate |
| | which will conclude when a | Limiting Volume |
| | particular volume is reached. | |
| | Volume Limits and phase | |
| | delivery volumes are involved | |
| | in the calculation of the limiting | |
| | volume for this phase. | |
| DPT_TIME | Describes a phase in which no | Duration |
| | fluid is delivered, but instead a | |
| | time delay is experienced. This | |
| | phase is used for Delayed Starts | |
| | and for Intermittent Volume | |
| | Over Time (IVOT) delay. | |
| DPT_IVOT | Describes a phase of delivery | Delivery Rate |
| | which will oscillate between | Limiting Volume |
| | delivering and not delivering, | Duration |
| | over repetitive time intervals, | PVD |
| | indefinitely. This can correspond | Total Duration |
| | to an IVOT infusion profile of a | State |
| | particular pump. | |
| DPT_END | This entry marks the end of | None |
| | delivery. A DPT_END entry will | |
| | be the last filled entry in the | |
| | table, even for infusions which | |
| | are continuous. | |

In an embodiment, referring again to FIGS. 4-5 and for example, 208, 210, 212, and check 214, during the traversal of the appropriate data structure holding the ordered collection of delivery phases, a data structure such as DC_PROGRESS can be used to accumulate time and de-accumulate volume. The calculation can be performed by initializing the DC_PROGRESS structure and passing it, along with successive entries from the Delivery Phase Table, to an accumulator function (the Add( ) function in the “class,” Reservoir Volume Time Remaining Accumulator in FIG. 5). The accumulator function returns a status to the calculation engine upon each call. According to an embodiment, the status returned to the calculation engine is defined by Table 2 below.

TABLE 2
Delivery Calculator Status Values

| Status | Description |
|---|---|
| DCS_CONTINUE | The phase entry was accepted. The calculation is |
| | not yet complete. Ready for the next phase entry. |
| DCS_COMPLETE_SUCCESS | The phase was accepted. The calculation is |
| | complete. The accumulated time in the |
| | DC_PROGRESS structure now holds the value of |
| | Reservoir Volume Time Remaining. |
| DCS_COMPLETE_FAIL | The calculation is complete. The accumulator |
| | determined from the phase that Reservoir Volume |
| | Time Remaining has no value at this time. |

In an embodiment, referring again to FIGS. 4-5, the Reservoir Volume Time Remaining Accumulator handles phase table entries according to Table 3 below.

TABLE 3
Reservoir Volume Time Remaining Accumulator Actions for Each Phase

| Phase Type | Actions |
|---|---|
| DPT_INIT | It is unexpected that the accumulator will ever |
| | encounter an entry of this type. If it does, the |
| | accumulator returns DCS COMPLETE FAIL. |
| DPT_CONTINUOUS | Since this phase type goes on indefinitely, the rest |
| | of the reservoir will be expended when in this |
| | phase. The accumulator calculates how much time |
| | it will take to empty the reservoir at this phase's |
| | delivery rate, adds it to the accumulator, and |
| | returns DCS_COMPLETE_SUCCESS. |
| DPT_VOLUME | The remaining reservoir volume is compared with |
| | the phase's limiting volume. If the reservoir will be |
| | expended before the end of the phase, the time to |
| | empty the reservoir is calculated and added, and |
| | DCS_COMPLETE_SUCCESS is returned. If the phase |
| | will complete before the reservoir is expended, the |
| | entire time for the phase is accumulated, and |
| | DCS_CONTINUE is returned. |
| DPT TIME | The entry's time duration is accumulated and |
| | DCS_CONTINUE is returned. |
| DPT_IVOT | The time to deliver the remaining volume at the |
| | specified delivery rate is calculated and added to |
| | the accumulated total. Additionally, the time spent |
| | in delay (the off-cycle portions of the oscillating |
| | delivery) is calculated and added to the total. |
| DPT_END | Encountering this entry implies that the infusion |
| | completes before the reservoir is empty. |
| | DCS_COMPLETE_FAIL is returned. |

According to embodiments, a number of assumptions regarding an inability to accurately predict the future can be incorporated according to the algorithms considered. For example, one assumption can be that no future interruptions of the infusion (via [STOP] key press, high-priority alarm, or other stimulus) are anticipated. For example, in evaluating what is known at the moment, it cannot be determined with certainty whether a future interruption will occur. But, in another example assumption, if an infusion is currently interrupted, the value of Reservoir Volume Time Remaining will comprise the amount of time it will take to deliver the rest of the reservoir volume once the infusion is resumed.

In another example assumption, no future changes in the infusion (Bolus or Loading Doses not yet programmed, Rate Changes, etc.) are anticipated. For example, in evaluating what is known at the moment, it cannot be determined with certainty whether a future change in the infusion will occur.

Further, until Flush Setup is begun, flush delivery is not anticipated; flush delivery is separate from the rest of the infusion. Because, regarding flush, the time of flush is never accurately known until it has been programmed In another example assumption, if an infusion has not yet started, or is complete, Reservoir Volume Time Remaining does not have a value. For example, if an infusion is not running, there is nothing to count down or decrement.

In another example assumption, if an infusion will complete before the reservoir is empty, then Reservoir Volume Time Remaining does not have a value. For example, Reservoir Volume Time Remaining is “meaningless” since the reservoir was not intended to be fully used. In embodiments, additional or fewer assumptions are used, according to the embodiment of the algorithms utilized.

In another embodiment, the aforementioned architecture comprises the framework to calculate myriad other metrics about or for an infusion pump or an infusion pump system. By defining additional accumulator functions, as represented by the “Infusion Time Remaining Accumulator” class in FIG. 5, additional infusion metrics can be calculated.

For example, an “Infusion Time Remaining” metric can be calculated to provide a measure of time before the infusion completes. As described above, an “Infusion Time Remaining Accumulator” is depicted in FIG. 5. In embodiments, the Infusion Time Remaining Accumulator is an additional accumulator which can be utilized in a way substantially similar to the Reservoir Volume Time Remaining Accumulator. The Infusion Time Remaining Accumulator is used to calculate the amount of time remaining until the infusion is complete, through all currently programmed phases of delivery.

According to embodiments, a number of assumptions regarding an inability to accurately predict the future can be incorporated according to the algorithms considered. For example, one assumption can be that no future interruptions of the infusion (via [STOP] key press, high-priority alarm, or other stimulus) are anticipated. For example, in evaluating what is known at the moment, it cannot be determined with certainty whether a future interruption will occur. But, if the infusion is currently interrupted (paused), then the Infusion Time Remaining can be calculated to provide an amount of time remaining until the infusion is complete, once the infusion has been resumed. In another example, if multiple reservoirs will be expended in the course of completion of the infusion, then the time necessary to perform the switching, changing, or replacement of reservoirs is unknown, and not accounted for (that is, it is assumed to take no time at all).

In embodiments, the Infusion Time Remaining Accumulator accumulates the time for each successive phase of delivery. For example, the calculation of Infusion Time Remaining progresses similarly to Reservoir Volume Time Remaining. In embodiments, the Phase Entry Table is generated. Further, and referring to Table 4 below, the calculation engine passes successive entries from the Phase Entry Table (along with a progress state object) to the Infusion Time Remaining Accumulator until the Infusion Time Remaining Accumulator returns a status other than DCS_CONTINUE. If the final status from the Infusion Time Remaining Accumulator is DCS_COMPLETE_SUCCESS, the calculation engine retrieves the final calculated result from the progress state object. If the final status from the Infusion Time Remaining Accumulator is DCS_FAILURE, the calculation engine concludes that the calculated delivery metric has no value under the current circumstances.

TABLE 4
Infusion Time Remaining Accumulator Actions for Each Phase

| Phase Type | Actions |
|---|---|
| DPT_INIT | It is expected that the accumulator will not encounter an entry |
| | of this type. If it does, the accumulator returns |
| | DCS_COMPLETE_FAIL. |
| DPT_CONTINUOUS | Since this phase type goes on indefinitely, the length of the |
| | infusion is theoretically infinite. Therefore, if this type of entry |
| | is encountered in the Phase Entry Table, then Infusion Time |
| | Remaining is treated as having no value. When the accumulator |
| | encounters an entry of this type, it returns |
| | DCS_COMPLETE_FAIL |
| DPT_VOLUME | The time to deliver the phase's limiting volume is added to the |
| | accumulated time and DCS_CONTINUE is returned. |
| DPT_TIME | The entry's time duration is added to the accumulated time and |
| | DCS_CONTINUE is returned. |
| DPT_IVOT | Therefore, if this type of entry is encountered in the Phase Entry |
| | Table, then Infusion Time Remaining is treated as having no |
| | value. |
| DPT_END | Encountering this entry implies that all phases of delivery have |
| | been accounted for, and the infusion will, in fact, complete. The |
| | accumulator returns DCS_COMPLETE_SUCCESS. |

In another example, a count of the number of additional similar reservoirs necessary to complete the entire infusion can also be calculated by similar algorithms.

As described above, embodiments of the Delivery Phase Table can be implemented such that it represents known delivery actions from the current moment forward. In another embodiment, the Delivery Phase Table (“DEL_PHASE_TABLE” in FIG. 5) can be implemented such that it describes all the phases of the infusion from the beginning to completion, and not just from the current moment forward. For the purposes of the calculation of Reservoir Volume Time Remaining, a “cursor” data object can thereby be derived to depict progress through the table. In embodiments of the Delivery Phase Table (“DEL_PHASE_TABLE” in FIG. 5), the Table can be used as the main representation of the programming of the pump, and can be used to drive delivery throughout the infusion, rather than being used solely as a passive calculation component. Further, delays built into an infusion sequence can be accounted for, such as preprogrammed delays to start or delays during infusion.

Various embodiments of systems, devices, and methods have been described herein. These embodiments are given only by way of example and are not intended to limit the scope of subject matter hereof. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized commensurate with the scope of subject matter hereof.

Persons of ordinary skill in the relevant arts will recognize that subject matter hereof may comprise fewer features than illustrated in any individual embodiment described above. The embodiments described herein are not meant to be an exhaustive presentation of the ways in which the various features of subject matter hereof may be combined. Accordingly, the embodiments are not mutually exclusive combinations of features; rather, the subject matter hereof may comprise a combination of different individual features selected from different individual embodiments, as understood by persons of ordinary skill in the art. It is also to be appreciated and understood that subject matter hereof could be adapted to accommodate an infusion regime utilizing multiple reservoirs that feed into a single line. For example, if an infusion was diluted in-line with saline or a drug flow included saline, embodiments could be adapted to make determinations of when the infusions of the drug and saline, respectively, would be completed. Further, it is to be appreciated and understood that subject matter hereof could readily account for various steps or phases of an infusion or planned infusion(s) (regardless of infusion type or method, such as a “tapered infusion”) and precisely determine the reservoir volume time remaining, among other metrics.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim.

1. A method of determining a delivery metric for an infusion pump, the method comprising:
determining at least a plurality of delivery phases of the infusion pump;
ordering the determined delivery phases;
traversing the ordered delivery phases by evaluating a phase characteristic for each delivery phase against a pump characteristic; and
determining the delivery metric based on the traversal of the ordered delivery phases.

2. The method of determining a delivery metric for an infusion pump of claim 1, wherein the phase characteristic for the delivery phase is a volume to be infused during the delivery phase, and the pump characteristic is a reservoir volume remaining, wherein evaluating a phase characteristic for each current and future infusion phase against a pump characteristic comprises:
subtracting the volume to be infused during the delivery phase from the reservoir volume remaining; and
adding the time to perform the delivery phase to a time accumulator.

3. The method of determining a delivery metric for an infusion pump of claim 2, wherein evaluating a phase characteristic for each delivery phase against a pump characteristic further comprises:
evaluating whether the reservoir volume remaining equals zero,
wherein when the reservoir volume remaining equals zero, presenting the time accumulator as the delivery metric.

4. The method of determining a delivery metric for an infusion pump of claim 2, wherein the delivery metric is at least one of reservoir volume time remaining, infusion time remaining, or number of reservoirs remaining.

5. The method of determining a delivery metric for an infusion pump of claim 1, wherein determining a plurality of delivery phases of the infusion pump comprises evaluating a programming parameter for the plurality of delivery phases.

6. An infusion pump system comprising:
at least one reservoir having a reservoir volume; and
an infusion pump including:
a pumping mechanism operably coupled to the at least one reservoir;
a processor including a memory and configured to control operation of the pumping mechanism; and
a calculation module configured to determine at least one characteristic about the at least one reservoir.

7. The infusion pump system of claim 6, wherein the calculation module is configured to determine at least one characteristic about the at least one reservoir by:
determining a plurality of current and future delivery phases of the infusion pump;
ordering the determined plurality of delivery phases;
traversing each of the ordered delivery phases by subtracting a volume to be infused during the delivery phase from a reservoir volume remaining, and adding the time to perform the delivery phase to a time accumulator,
wherein when the reservoir volume remaining equals zero, presenting the time accumulator as the at least one characteristic.

8. The infusion pump system of claim 6, wherein the infusion pump further includes a communication port, the system further comprising a network operably coupled to the communication port, wherein data about the pump can be transmitted to and from the network.

9. The infusion pump system of claim 6, wherein the ordered delivery phases are stored in memory in a table data structure.

10. An infusion pump comprising:
a pumping mechanism operably coupled to at least one reservoir;
a pump control subsystem including a processor and a memory and configured to control operation of the pumping mechanism, the operation including a plurality of delivery phases; and
a calculation module configured to:
determine the plurality of delivery phases,
order the determined delivery phases, and
traverse the ordered delivery phases by evaluating a phase characteristic for each delivery phase against a pump characteristic and determining a delivery metric based on the traversal of the ordered delivery phases.

11. The infusion pump of claim 10, wherein the phase characteristic for the delivery phase is a volume to be infused during the delivery phase, and the pump characteristic is a reservoir volume remaining, wherein evaluating a phase characteristic for each current and future infusion phase against a pump characteristic comprises:
subtracting the volume to be infused during the delivery phase from the reservoir volume remaining; and
adding the time to perform the delivery phase to a time accumulator.

12. The infusion pump of claim 11, wherein evaluating a phase characteristic for each delivery phase against a pump characteristic further comprises:
evaluating whether the reservoir volume remaining equals zero,
wherein when the reservoir volume remaining equals zero, presenting, on the infusion pump, the time accumulator as the delivery metric.
